(19) United States
(12) Patent Application Publication
(10) Pub. No.: US 2008/0200896 A1
(43) Pub. Date: Aug. 21, 2008

(54) Device and Method for Treating a Blood Vessel

(76) Inventors: Ascher Shmulewitz, Tel Aviv (IL); Zeev Brandeis, Rosh Ha'ain (IL)

Correspondence Address: Pearl Cohen Zedek Latzer, LLP, 1500 Broadway, 12th Floor, New York, NY, 10036, US

(21) Appl. No.: 11/632,641

(22) PCT Filed: Jul. 13, 2005
(86) PCT No.: PCT/US2005/024657

§ 371 Date: Dec. 18, 2007

Related U.S. Application Data
(60) Provisional application No. 60/587,873, filed on Jul. 15, 2004.

(51) Int. Cl. A61M 25/10; A61M 25/09

(52) U.S. Cl. 604/500; 604/103.07; 604/99.01; 604/99.02

(57) ABSTRACT

Device and method for treating a vessel including for example the insertion of a catheter toward a target destination using a guidewire. The catheter may include for example a proximal balloon, being shaped to enable the formation of for example a funnel shape when infated, and a distal balloon. The target destination, for example, a thrombosis, chronic total occlusion or myocardial infarction area may be substantially enclosed by the proximal balloon and the distal balloon.

### FIELD OF THE INVENTION

The present invention relates to methods and devices for treating, for example, thrombosis, chronic total occlusion or myocardial infarction. Specifically, embodiments of the present invention relate to methods and apparatuses used in percutaneous angioplasty for treating or opening of vascular occlusions.

### BACKGROUND OF THE INVENTION

Abnormal thrombosis may occur in any vessel at any location in the body. The principal clinical syndromes that may result are acute myocardial infarction (MI), deep vein thrombosis, pulmonary embolism, acute no hemorrhagic stroke, acute peripheral arterial occlusion, and occlusion of indwelling catheters. Other syndromes may also occur.

One common treatment of thrombosis may be fibrinolytic therapy. However, common hemorrhagic problems seen after fibrinolytic therapy may include gastrointestinal bleeding, retroperitoneal bleeding, pericardial bleeding, genitourinary bleeding, epistaxis, ecchymosis, gingival bleeding, and bleeding from puncture sites. Large hematomas at peripheral arterial puncture sites occasionally may cause a compartment syndrome. Often the most dreaded complication of fibrinolysis may be intracranial hemorrhage, but serious hemorrhagic complications may occur from bleeding at any site in the body. Overdoses of fibrinolytic agents may cause severe hemorrhagic complications.

In patients receiving fibrinolysis for MI, for example, the overall incidence of hemorrhagic complications is approximately 10%, and the incidence of intracranial hemorrhage is about 0.7%. In patients receiving fibrinolysis for acute stroke, the incidence of intracranial hemorrhage may be higher In patients receiving fibrinolysis for acute stroke, any sign of a major infarct increases the likelihood of hemorrhagic conversion.

Despite the increased risk of hemorrhage in patients with a massive stroke, fibrinolysis remains indicated whenever other exclusion criteria may be absent because the potential benefit may be tremendous in this population of patients, who almost always have a dismal outcome if therapy is withheld.

Chronic total occlusion (CTO) of a coronary artery is the complete obstruction of the vessel aged at least one month according to most researchers or three months according to others. The term may be also used to describe total occlusions with TIMI grade 1 flow, the so-called “functional occlusions”, which may be defined as the presence of only faint, late anterograde flow in the absence of a discernible lumen. These occlusions generally represent 5% to 15% of the total number of angioplasty procedures of a cath lab activity and this percentage depends on the selection of the cases and the experience of the staff. Successful recanalization of a chronic total coronary occlusion remains even today, 20 years after the first angioplasty procedure was performed, a challenge for interventional cardiologists. The most significant limitation to successful recanalization may be the failure of the guide wire to cross the lesion.

The occluded segment of a vessel lumen generally includes two types of tissue: atheromatous plaque and old thrombus. Two causal pathogenetic phenomena are generally implicated: (a) the late organization and development of an acute occlusion (involving a large area of an old thrombus), generated by atheromatous plaque rupture, which is usually located at the distal end of the minimum lumen diameter and (b) the progressive occlusion of a long-term high-degree stenosis (involving a large area of atherosclerotic plaque and quite often additional layers of plaque surface thrombus). The occlusive mass is mainly composed of fibrous and mixed components including a small quantity of cholesterol, which progressively decreases with time and is replaced by dense collagen and calcium deposits. Usually the plaque fibrous cap (which may be the hardest part of the plaque) is located at both ends of the occlusion. The intraluminal process is often accompanied by a negative remodeling (vessel shrinkage) of the artery, which is mainly observed in chronic occlusions older than 3 months. The negative remodeling process is generally connected to the replacement of soft plaque tissue with fibrous tissue, mainly in the middle section of the occlusion. The importance of the replacement of these elements may be greater in total occlusions of small length. In larger occlusions, the thrombus organization is responsible for the presence of a soft inner core in the center of the occlusion which may be also the targeted most vulnerable point during the attempted transluminal perforation procedure. The angiographic morphology of the occlusive lesion may close abruptly with almost a complete absence of vessel stump, can display a stump with progressive narrowing of the lumen or can include formation of neovessels. The frequency of neovessel formation progressively increases in proportion to the duration of the occlusion (reaching 85% in occlusions older than a year). Neovessel formation is not affected by the length of the occlusion while there are conflicting views with respect to the existence of links between these vessels. Some publications report a connection between neovessels and the formation of neovascular channels, whose presence contributes in a positive way in cases of attempted transluminal recanalization, while others report the existence of links between neovessels and the vasa vasorum of the adventitia, which negatively affect the advancement of the guide wire through the obstruction and predispose to the development of complications. Both types of links may coexist in the majority of cases

Coronary angioplasty of CTOs may be associated with specific limitations. As expected, the primary success rate may be lower, while expenses (quantity of used materials and contrast) and radiation exposure to both patients and physicians associated with angioplasty of occluded coronary arteries may be increased, compared to angioplasty of subtotal occlusions. Failure in most cases may be due to unsuccessful crossing of the guide wire and less often to a failure of balloon insertion through the obstruction. Even in cases of initial vessel recanalization, the presence of local thrombus may lead to peripheral embolization of thrombotic material or even plaque debris resulting in slow or lack of angiographic flow through the vessel (no reflow phenomenon). CTOs that are successfully opened show a higher rate of angiographic restenosis despite technological advances and increased operator experience. Therefore, the nature of the lesion and the “more traumatic” materials used, as well as the coronary substrate in which they are used, are associated with a lower initial success rate than angioplasty of arteries that are stenotic but not occluded. Thus the careful selection of patients and angioplasty materials becomes a major determinant for the successful and safe completion of the procedure.

It may be generally accepted that to adequately justify an attempt of coronary artery CTO recanalization the following conditions should apply: (a) presence of serious angina despite appropriate medical treatment, a fact which may be linked to the existence of a large area of viable and active myocardium, (b) anticipation that the reperfusion of the hibernating myocardium may improve the partial and total left ventricular contractility, at the same time limiting its remodeling, which may be an important factor of unfavorable long term clinical outcome (“open artery hypothesis”) and (c) hope that the restoration of the obstructed vessel patency may aid in the development and provision of collateral circulation towards the contralateral artery in case the latter becomes occluded at a latex stage.

In patients with single-vessel disease, the indication for recanalization may be a combination of its functional importance in causing severe ischemia and limiting the physical activity of the patient who is receiving full medication on the one hand and, on the other hand, of the presence of favorable angiographic characteristics. The absence of the latter may not be a strictly forbidding criterion in cases where the patient's functional capacity is severely limited despite an optimum medical treatment. In patients with multivessel disease, CTO recanalization rarely constitutes the target-lesion, but it may be included in the intervention sites for full revascularization. In these cases, the inability to reopen the occluded segment or the existence of particularly unfavorable angiographic characteristics may be the main reasons for selecting surgical intervention as the treatment of choice. Clearly this strategy may be modified if a total reperfusion of the patient is not considered to be mandatory.

In cases of CTO angioplasty all the known limitations for the success of this method apply: location in the circumflex artery or in the distal segments of the coronary vessels, presence of marked tortuosity and bends in the target-artery as well as calcifications proximal to or at the lesion site. There are however additional specific factors that determine the chance of successful recanalization: The origin of a lateral branch at the site of occlusion may be an important adverse factor (success rate 32% versus 83% in the absence of such). A minimum distance of 2 mm may be necessary between the origin of the lateral branch and the site of occlusion; The presence of bridging collaterals towards the distal segment characterizes occlusions with a duration of at least 2 months (late angiogenesis) and constitutes an adverse factor for the success of the intervention, as well as a risk factor for possible vessel perforation. The angiographic image of the stump carries a predictive value. A progressively narrowing diameter represents a particularly favorable morphology while, on the other hand, a rounded-ends diameter has unfavorable prognosis. A central course of the stump lumen may be also a favorable factor; the length of the occlusion may be a major determinant of success and many researchers consider a length of 15 mm to be a critical success limit especially if a curve is involved. Functional occlusions may be caused by the existence of a very small diameter in the absence of an angiographically discernible lumen, usually display a higher success rate or may be associated with the development of neovessels, indicative of an old and well-organized CTO.

CTO angioplasty may consist of the following individual objectives: (1) perforation of the total occlusion with the use of a guide wire and advancement of the guide wire to the distal segment of the vessel, (2) dilatation of the underlying lesion or removal of occlusive material in order to restore the patency of the lumen and (3) preservation of the patency of the recanalized vessel by the administration of medication and the implantation of intracoronary stents.

The inability to cross the CTO lesions with a guide wire may be the principal cause of failure (>50%) of the intervention. In cases of suboccluded lesions angioplasty, the advancement of a soft wire through the lesion is usually easy and fast. During an attempted recanalization of a CTO, the guide wire may pass through the initial usually hard and fibrous segment of the lesion, advance to the central core of the occlusion which may be composed of elements of varying degree of hardness, being careful not to steer it in a subendothelial course, and finally perforate the distal segment of the occlusion, which usually consists of hard fibrous material. Thus, the special characteristics which determine the choice of a guide wire may be: its hardness, the ability to direct it within the occlusion, the ability to safely perforate without taking a subendothelial course and the characteristics of reduced friction at its distal end.

Several types of guide wires may be available for CTO angioplasty, including wires of varying diameter, length and distal end morphology, with special polymer coating that may increase its sliding ability and the ability to direct it within the occlusion. The use of “over-the-wire” angioplasty catheter systems or special thin catheters (multifunctional probing) may be recommended to adequately support the wire during the attempt to perforate the total occlusion and to maintain its central position within the occluded segment and away from the vascular wall.

In the first historic CTO angioplasty series which included a mixed population with occlusions of a mean duration 1-4 months, a successful recanalization was reported at a rate of 42% to 63%. This rate decreased as the duration of occlusion increases and a rate of 11% to 55% was reported in the angioplasty of CTOs aged >6 months. More recent publications also report, despite the technological improvement of the materials used, a persistent low initial success rate of the method in CTO cases, especially when those cases have adverse features (39% to 56%/).

Serious research attempts at designing new wires or techniques have not led to a significant improvement of the results, while they have increased the cost and the percentage of preoperative complications, and as a result their use and especially that of laser wires, has almost been abandoned in clinical practice. The effectiveness of the usage of laser wires, even the latest generation, in the recanalization of chronic total occlusions did not appear to be superior to the usage of conventional wire, according to the results of the TOTAL study (see “Recanalization of Chronic Total Occlusions: Basic Techniques and New Guide Wires”, by J. Kahler, R. Koster, C. W. Hamm, Dept. of Cardiology, University Hospital Hamburg, Hamburg, Germany).

A new CTO angioplasty technique with an increased success rate was developed by L. K. Michalis et al., called angioplasty using “vibrating wires”. According to this new technique, the proximal end of a common guide wire within an “over the wire” catheter may be connected to a portable electrically-driven device which is powered by battery. The activation of this device causes a slight vibration along the longitudinal and transverse axis of the wire at a frequency of 16-110 Hz. The wire movement may be transmitted through the catheter and causes a complex movement of the distal end of the wire in the same axes. The degree and range of the distal end movement depends on the response frequency and the length of the wire that protrudes from the end of the angioplasty catheters. With this technique it may be possible to advance the wire within the occlusion through the most pliable regions, while securing its intraluminal course. The investigators report successful recanalization and finally successful CTO angioplasty at a rate of 85.9% and 75.6% respectively in a patient population with particularly adverse characteristics, such as chronic lesions (aged >6 months in >80% of the patients), presence of bridge collaterals in 42% and occlusion length >15 mm in 69.2%, with a small percentage of serious preoperative adverse events.

Site-specific delivery may enable a therapeutic concentration of a drug to be present at the desired target without exposing the entire body of the patient to a similar dose. The precision, miniature size and performance characteristics of the delivery system may allow for continuous site-specific delivery to a variety of precise locations within the body.

Local or site-specific delivery of therapeutic agents increases drug concentration at the tumor target, decreases systemic exposure and toxicities, and increases the duration of exposure of the target tissue to the drug. Experimental and clinical studies have demonstrated statistically significant increases in survival associated with local therapy for brain tumors. Drugs have been delivered via controlled-release biodegradable matrices and infusion pumps.

Recently, researchers in Canada showed the use of chelating agents and collagenase in an animal model to disrupt chronic total occlusions. The drug was given locally using an intra-arterial injection. The success rate was 82%.

A common method of treatment of DVT may be catheter-directed lysis. This treatment calls for inserting a catheter directly at the site of the thrombus and infusing a lytic agent. This method may have the advantage of infusing the lytic agent directly into the thrombus, but it also entails a lengthy hospital stay. Catheter-directed lysis may also have the potential to only partially dissolve thrombus, causing pieces of thrombus to travel downstream.

Because catheter-directed lysis may generally require large amounts of lytic agent infused over a long period of time, the risk of bleeding complications, including potentially fatal cerebral hemorrhage increases dramatically. Further, catheter-directed lysis treatment may pose questions with lytic dosing, timing and duration. Finally, long-term studies in post-lysis patients with ilio-femoral DVT have demonstrated that muscle pump function and valvular incompetence are severely compromised in approximately 95% of the patients at five-year follow up, despite an improvement in venous outflow.

In recent years, less invasive catheter-directed dissolution systems of thrombus in the vasculature have been developed. Additionally, significant attention has been focused on identifying the most effective type, delivery and dose of the dissolution medication. Multiple clinical studies have been initiated and have produced key recommendations. Some of the key recommendations include: Definition of acute (<14 days) versus chronic (>14 days) thrombus formation; Definition of successful and failed thrombolysis along with a grading system; Comparison of the effectiveness of varied infusion routes; Medication dosages and durations; Systemic infusion versus intrathrombic bolusing versus intrathrombic lacing.

Consistently, the publications surrounding this clinical focus may have stated that saturating and regionalizing the dissolution medication within the thrombus may be paramount to achieving recanalization and decreasing hemorrhagic complications. It may also afford the clinician the immediate opportunity to identify the underlying lesion and correct it, using either an endovascular or open technique.

The combination of therapeutic modalities, specifically medication and a catheter-based delivery system, appears to be an effective course of treatment for the patient. Catheter-based delivery systems have been effectively utilized for over thirty years. The risks, benefits and dosing of thrombus dissolving medications have been widely discussed and published.

Despite all the advances in percutaneous coronary intervention, there remains a need for a cost effective, easy-to-operate device to treat acute and chronic total occlusions while minimizing the risk of embolization and late complications, such as restenosis.

In peripheral areas of the human body, particularly legs and feet, bi-leaflet valves exist within veins to assist in returning blood to the heart. These valves restrict blood flow in the retrograde direction (opposite normal blood flow) by acting as a check valve, closing when sufficient normal blood flow to keep them open is absent.

### BRIEF DESCRIPTION OF THE DRAWINGS

The principles and operation of the system, apparatus, and method according to the present invention may be better understood with reference to the drawings, and the following description, it being understood that these drawings are given for illustrative purposes only and are not meant to be limiting, wherein:

FIG. 1 is a schematic illustration of a catheter device, according to some embodiments of the present invention;

FIG. 2 is a schematic illustration of a catheter device with two inflated balloons adjacent to a target area, according to some embodiments of the present invention;

FIG. 3 is a schematic illustration of a catheter device with two deflated balloons, according to some embodiments of the present invention;

FIG. 4 is a schematic illustration of a pumping mechanism, according to some embodiments of the present invention;

FIG. 5 is a schematic illustration of a catheter device with inflated balloons surrounding debris, according to some embodiments of the present invention; and

FIG. 6 is a flow chart describing a method of treatment, according to some embodiments of the present invention.

It will be appreciated that for simplicity and clarity of illustration, elements shown in the drawings have not necessarily been drawn to scale. For example, the dimensions of some of the elements may be exaggerated relative to other elements for clarity. Further, where considered appropriate, reference numerals may be repeated among the drawings to indicate corresponding or analogous elements throughout the serial views.

### DETAILED DESCRIPTION OF THE INVENTION

The following description is presented to enable one of ordinary skill in the art to make and use the invention as provided in the context of a particular application and its requirements. Various modifications to the described embodiments will be apparent to those with skill in the art, and the general principles defined herein may be applied to other embodiments. Therefore, the present invention is not intended to be limited to the particular embodiments shown and described, but is to be accorded the widest scope consistent with the principles and novel features herein disclosed. In other instances, well-known methods, procedures, and components have not been described in detail so as not to obscure the present invention.

Embodiments of the present invention may enable treatment of a target destination in or proximal to a blood vessel, including opening of a vessel(s), providing pharmaceutical agents to a vessel(s) or target area(s), and implementing other suitable treatments or procedures to a vessel(s) or selected parts of a vessel(s). A catheter device may be used, for example, to enable a physician to treat a vascular occlusion, by isolating a treatment region, break down an occlusion by means of, for example, a low-frequency vibrating pump and/or an infused physician-specified fluid, and remove at least elements of the occlusion. A device or method according to some embodiments may simultaneously prevent embolization prior to or during percutaneous angioplasty. In one embodiment a device may be inserted and advanced through, for example, the venous system and into the vein in a retrograde direction by opening a valve or a vessel with a fluid, enabling passage of the device through the valve or the vessel. The fluid may provide pressure against the valve and/or the surrounding vessel wall, thereby opening the cusps of the valve and allowing passage through the valve.

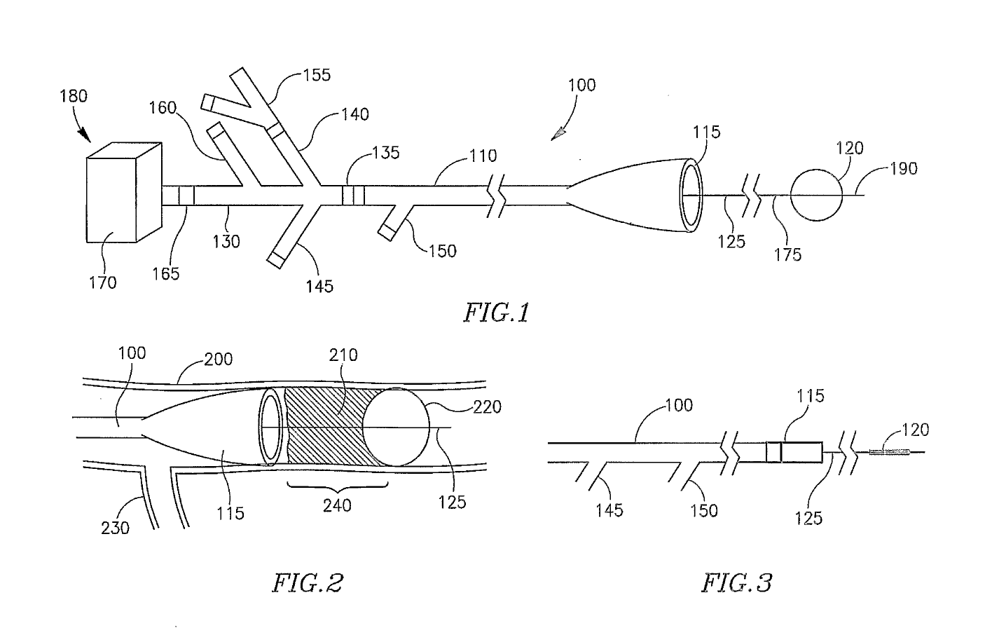

Reference is now made to FIG. 1, which is a schematic illustration of a catheter device 100, according to some embodiments of the present invention. Catheter device 100 may include catheter 110, which may be, for example, a multi-lumen coaxial catheter. Catheter device 100 may include a proximal end 180 and a distal end 190. Proximal and distal when use herein ate relative terms, typically relative to the control end or holding end of catheter device 100. The control end (e.g. the proximal end) may be used for holding and operating catheter device 100, for example, by a doctor or a health professional. Catheter 110 may include a proximal balloon 115, which may be relatively near, to the holding end or proximal end 180. Catheter device 100 may include and a distal balloon 120, which may be relatively distant to the holding end or proximal end 180, for example, relatively neat to the target area (e.g., the distal end), and/or conventional or special-purpose guidewire 125. Guidewire 125 may be moveable inside catheter 110 and may be able to move freely or semi-freely. Catheter device 100 may further include pusher handle 130, vibration mechanism 170, and connector 135, for connecting catheter 110 to guidewire 125, vibration mechanism 170, pusher handle 130 etc. Pusher handle 130 may be used for controlling, for example, pushing and pulling of guidewire 125 in relation to catheter 110. Catheter 110 may be associated with a plurality of ports, for example, ports 145 and 150 for deflating the respective balloons, port 160 for controlling guidewire 125, port 140 for inserting and/or suctioning out substances (e.g., administering drugs, extracting debris etc.), and port 165 for connecting to vibration mechanism 170. Catheter device 100 may be associated with a drug dispensing mechanism 155. Drug dispensing mechanism 155 and port 140 may enable the entry of drugs or pharmaceutical agents or fluids directly to a treatment site or region (e.g., luminal segment, a valve), for example, to accelerate the passage of guidewire 125, and/or the dissolving, disintegrating, fragmenting, decomposing, opening or decaying etc. of a target occlusion etc. Other numbers and/or types of ports may be used.

Proximal balloon 115, which may be for example a compliant or non-compliant balloon, may be connected to catheter 110, and may be located substantially near the proximal end 180 of catheter 110. Proximal balloon 115 may be shaped to enable formation of a funnel shape or a container when inflated. For example, the proximal balloon may be shaped in a funnel form or cone shaped when inflated, or may have other suitable shapes. Proximal balloon 115 may have flexible distal end to allow a broader and wider distal end when inflated. For example, when proximal balloon 115 is inflated the distal end, relatively close to a target area or an occlusion may become broader than its proximal end, which is relatively close to the holding point of the catheter, to allow proximal balloon to be in proximity to the target area or the occlusion. Such an embodiment may be used for the treatment of CTO; however other conditions may be treated. Proximal balloon 115 may have a cone or funnel shape or another suitable shape such that the volume or area relatively near to proximal end 180 may be smaller and/or narrower than the volume or area relatively neat to distal end 190. A funnel or cone shape may be used, but other shapes allowing for one end being larger or wider may be used. Proximal balloon 115 may have an orifice to enable collection of debris or loosened elements from an occlusion, fluids etc. Other types of balloons may be used For example, in CTO treatment catheter 100 or proximal balloon may be used for dragging and/or collecting unwanted elements (e.g. occlusive material or debris from a partially disintegrated occlusion).

Distal balloon 120 may be mounted on a small profile shaft, which may be constructed on guidewire 125, or on an attachable shaft 175, substantially near the distal end of guidewire 125. For example, a guiding wire with an expandable balloon may be used, for example, “GuardWire” manufactured by Medtronic Inc. (http://www.medtronic.,com/medtronic_vascular/dp_guardwire.html. 710 Medtronic Parkway, Minneapolis, Minn. 55432). In some embodiments balloons 115 and 120 may be individually controlled, and may thereby be located at selected locations. For example, balloons 115 and 120 may be positioned at selected distances from each other on both sides of an occlusion to be treated. Balloon 120 may be non-permeable, and may help create a vacuum between balloon 115 and balloon 120, or may be permeable or semi-permeable, for example, to function as a filter.

When proximal balloon 115 and distal balloon 120 are inflated, as can be seen with reference to FIG. 2, a treatment area, region or zone 240 may be isolated, for example, to enable treatment of a selected luminal segment, for example, by maintaining a controlled concentration, volume and pressure of fluid or other substances infused by catheter 110 into a lumen. Proximal balloon 115 and/or distal balloon 120 may allow for the anchoring of a vessel 200. For example, balloon 115 may create a vacuum to prevent the dislodging of debris from occlusion 210, and may apply force on the vessel wall 200 at the side of the occlusion 210. Proximal balloon 115 and distal balloon 120 may be controlled to enable evacuation of unwanted elements from inside catheter 100 or balloon 115 etc. Catheter 110 may be used to treat diseases other than CTO.

According to one embodiment of the invention catheter 110 may include a proximal balloon 115, and a conventional or special-purpose guidewire 125. Proximal balloon 115 may be relatively near to the holding end or proximal end 180, and may enable formation of a funnel shape when inflated. For example, when proximal balloon 115 is inflated the distal end, relatively close to a target area or an occlusion may become broader than its proximal end, which may be narrower than its distal end, to allow proximal balloon to be, for example, in proximity or relatively close to the target area or the occlusion. For example, the distal end of balloon 115 may be more flexible than the proximal end to allow expansion in the distal end neat the target area or the occlusion. Guidewire 125 may have a stiff tapered or wedge shaped tip which may be used for loosing occlusion elements in order to extract them from the vessel. Devices including tapered or wedge shaped tips may be provided by for example Brivant Medical Engineering, www.brivant.com. Guidewire 125 tip may be inflexible, solid and firm to allow for example cracking of the occlusion. A tapered or a cone tip shape of guidewire 125 may be used, but other shapes allowing cracking of the occlusion may be used. Such an embodiment may be used for the treatment of CTO; however other conditions may be treated. Other suitable tip shapes or configurations may be used.

In some embodiments, for example, embodiments which may be used for the treatment of CTO, balloon 115 may be inflated at a selected location before occlusion 210, so as to enable occlusion 210 to act as a plug, thereby forming a treatment area, without the usage of a distal balloon.

According to some embodiments of the invention, catheter 100 may include a port, for example, port 140, which may administer insert or inject fluids or other agents to a specific area in the treating vessel to enable opening of valve or expending of a vessel, to allow inserting of catheter 100 into the vessel. For example, the blood flow through the valve may temporarily increase by injecting fluids through port 140 of catheter 100 proximal to the valve. The temporarily increased blood flow may open the cusps of the valve allowing passage of catheter 100 through the valve in the retrograde direction. In accordance with other embodiments of the invention catheter 100 may be inserted into a vessel, for example, a vein and subsequently advanced until, for example, distal end 190 of catheter 100 may be in a proximal position to a bi-leaflet valve. A rapid injection of fluids, for example, saline or other physiological fluid through port 140 may expend the vessel or may open the cusps of a valve, allowing catheter 100 to pass through the open valve or expended vessel. According to some other embodiments of the invention a syringe may be connected to one of catheter 100 ports and may be used to generate a temporary rapid blood flow in an antegrade direction through the vessel. By, for example, aspirating blood with the syringe, blood may flow through a valve whereby opening the cusps of the valve or expending the vessel walls. Catheter 100 may then be able to pass through the open valve or expended vessel.

As can be seen with reference to FIG. 3, catheter 100 may be coated with one or more thin film balloons 115, which may be subsequently inflated through a port, for example, port 145, which may be mounted on pusher handle 130. Guidewire 125 may be coated with one or more thin film balloons 120, which may be subsequently inflated through a port, for example, port 150, which may be mounted on pusher handle 130. Distal balloon 120 may be positioned distal to the anchoring proximal balloon 115, for example, in the range of 0.5 cm to 50 cm, or any other suitable distances apart, so as to enable a target occlusion that is to be treated to be substantially surrounded by balloons 115 and 120, to enable treatment of different clinical conditions. Guidewire 125 may be, for example, 0.014″ thick, or may have other suitable dimensions.,

According to some embodiments of the invention, vibration mechanism 170, for example a pump, may perform, for example, pumping actions to generate transluminal subsonic, sonic and/or ultrasonic vibrations, to generate vibrations in one or more frequencies. In some embodiments these vibrations may be used, for example, to help in dissolving, disintegrating, fragmenting, decomposing, decaying, breaking apart or weakening etc. a target occlusion, to enable dissolution and subsequent extraction of at least elements of the target occlusion. In some embodiments these vibrations may be used to cause guidewire 125 to vibrate, further helping the advancement of guidewire 125 in a lumen, for example, to penetrate an occlusion. In other embodiments these vibrations may be used to cause inserted substances, for example, fluids or other pharmaceutical agents, to vibrate, thereby aiding such an agent's treatment of a target area. Vibration mechanism 170 may enable generation of vibrations, for example, in single or multi-frequency mode, in the subsonic to ultrasonic range. A plurality of vibrations types at a plurality of frequencies may be used, to provide selected vibrations to one or more elements of catheter device 100, or to a selected destination. Such embodiments may be used for the treatment of CTO; however other conditions may be treated.

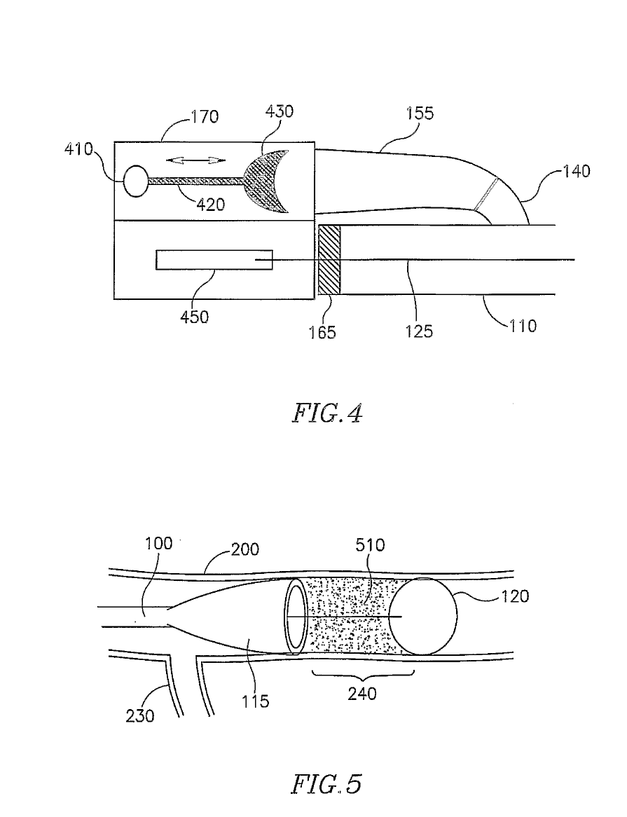

As can be seen with reference to FIG. 4, vibration mechanism 170, according to some embodiments of the present invention, may include a reciprocating pump 410 that works diaphragm 430 to deliver, for example, short, rapid pulses at high exit-jet velocity through catheter 100. For example, small volumes of drugs infused through port 140, such as thrombolytic enzymes, may be pushed by Vibration mechanism 170 into an enclosed segment of vessel 200 For example, entry of fluids from dispenser or container 155 may be aided using low-frequency vibrations generated by Vibration mechanism 170. For example, a pulsatile pumping action by Vibration mechanism 170 may enable forceful local pulsatile infusion of substances into a target region. For example, fibrinolytic enzyme, which has been shown to disrupt thrombi, increase clot surface area, and thereby hasten enzyme action, as compared with conventional constant infusion methods (see, for example, Kandarpa et al, Radiology, Vol 168, 739-744), may be infused by Vibration mechanism 170 to a selected occlusion. Infusion of advantageous substances to aid occlusion breakdown preceding treatment of thrombus may help shorten thrombolytic therapy, thereby reducing patient morbidity. Enhanced fibrinolysis may result from accelerated enzymatic degradation rather than from mechanical disruption of fibrin. The effects may be non-thermal and may be mediated in part by increasing transport of reactants into the fibrin matrix (see, for example, Siddiqi et al, Blood, Mar. 15, 1998).

Vibration mechanism 170, according to some embodiments of the present invention, may include mechanical rotational element 450, which may, for example, generate vibrations on guidewire 125 by rotating and/or otherwise moving around guidewire 125. For example, mechanism 450 may secure a proximal end of guidewire 125, and may rotate in alternate directions, and/or move forwards and backwards, or in other directions to generate, for example, sub-sonic high frequency vibrations along guidewire 125. Generated vibrations may aid the entry of guidewire into a vessel and/or through an occlusion etc.

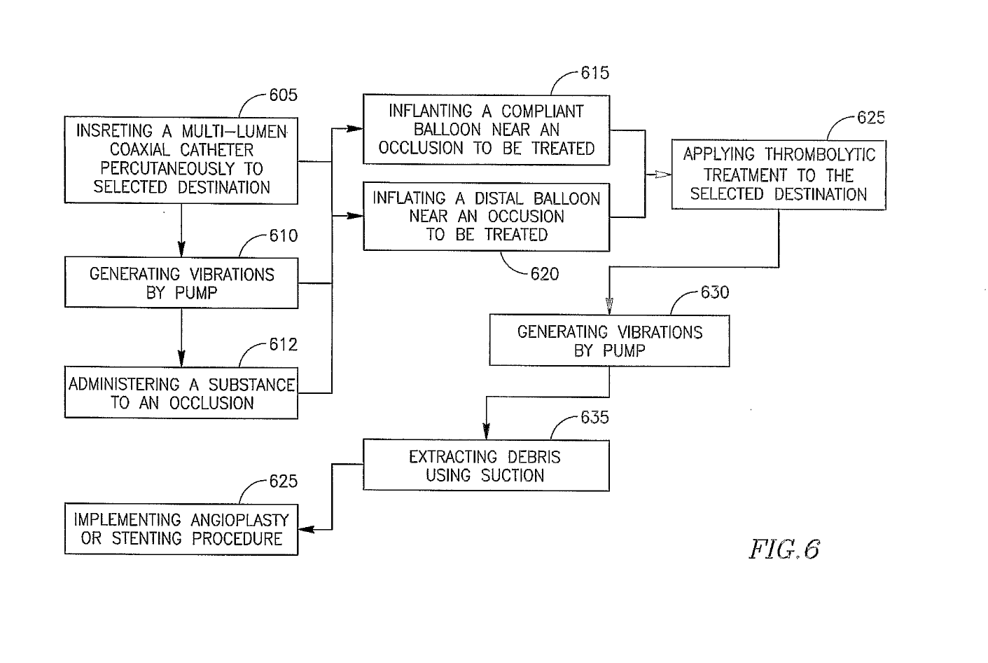

Reference is now made to FIG. 5, which illustrates a catheter with both balloons inflated according to an embodiment of the invention. Catheter 100 is shown, for example, following thrombolytic therapy that succeeded in breaking down an occlusion into loose debris 510. Distal balloon 120 may be partially deflated and may pull back debris 510 into proximal balloon 115. When the proximal and/or distal balloons are deflated, fox example, any remaining debris 510 may be trapped between the balloons and subsequently pulled back through the guidewire 125 and/or catheter 100. Additionally or alternatively, debris may be extracted from guidewire 125, catheter 100 and/or balloon 115 using a suction mechanism, for example, a syringe, at one or more ports. Other types of balloons, and other shapes of balloons, may be used. In order to treat CTO, the proximal balloon 115 may be inflated next to occlusion (for example, occlusion 210 of FIG. 2), to form, for example, a funnel-shape structure capable of receiving and holding debris 510. The inflated funnel may be pushed adjacent to occlusion 210 and may be anchored into vessel 200, while applying a radial force on the vessel 200, thereby facilitating the process of blunt dissection.

In some embodiments catheter device 100 may be used to treat target areas in or proximal to blood vessels, for example, to provide cancer therapy to a selected destination in a vessel.

In one embodiment balloon 120 may be at least partially extracted while inflated, for example through balloon 115, to enable dragging of debris 510 and/or occlusive material out of treatment area 240.

In one embodiment treatment for, for example CTO, may be implemented even where target destination (e.g., an occlusion) is in close proximity to branches in internal lumen. As can be seen in FIG. 5, the funnel shape of balloon 115 may enable extension of the distal end of balloon 115 to the area of occlusion 220 or debris 510, while leaving branch 230 substantially unblocked by balloon 115, to allow flow of blood to side-branch 230 during a procedure. Such an embodiment may be used for the treatment of CTO; however other conditions may be treated

In some embodiments of the present invention balloon 115 and/or 120 may be used to lead catheter 100 and/or guidewire 125 along vessel 200 and/or through occlusion 220. For example, catheter 100 may be extended into vessel 200 while balloons 115 and 120 are deflated, using guidewire 125 When guidewire 125 gets obstructed, for example, by a turn in vessel 200, balloon 115 and/or 120 may be inflated, thereby centering guidewire 125, to enable guidewire 125 to be positioned substantially towards the center of vessel 200. Balloon 115 and/or 120 may be subsequently deflated and/or inflated any number of times to help advance advancement into vessel 200.

According to some embodiments of the present invention, balloon 120 may be extracted from vessel 200 towards balloon 115, during or following treatment of an occlusion. For example, in the case where pharmaceutical agents have been applied to break down an occlusion, as the occlusion breaks down balloon 120 may be positioned closer to balloon 115 to enable minimizing of the size of the treatment area. This may enable the concentration of pharmaceutical agents in the treatment are to be increased, and may enable reducing the exposure time of elements of vessel 200 to the pharmaceutical agents.

Reference is now made to FIG. 6, which is a flow chart describing a method of applying thrombus treatment, according to some embodiments of the present invention In block 605 a catheter may be percutaneously entered or inserted into a lumen, to a target destination. The target destination may be, for example, an occlusion. Guidewire 125 may help advance catheter 100 into a target lumen. Catheter 110 may be advanced into a lumen such that proximal and distal balloons may respectively be located at at least two ends of a target destination, for example, an occlusion or other target. Other locations may be selected for lodging of the balloons. In block 610 vibration mechanism 170 may generate vibrations to aid guidewire 125 advances in catheter 100, for example, to penetrate an occlusion. In block 612 substances, for example pharmaceutical agents, may be applied to the occlusion to aid guidewire 125 advance in catheter 100, for example, to penetrate the occlusion. In block 615 proximal balloon 115 may be inflated near an occlusion to be treated, for example, relatively next to the holding end or proximal end 130 of catheter 100, also referred herein as a proximal end of a target destination, e.g., an occlusion. The proximal end of the target may be closer to the control end of the catheter and the distal end may be further from the control end. Such an embodiment may be used for the treatment of CTO; however other conditions may be treated. In block 620 distal balloon 120 may be inflated near an occlusion to be treated, for example, relatively distant to the holding end or proximal end 130 of catheter 100, also referred to herein as a distal end of a target destination, e.g., an occlusion, such that the occlusion to be treated may be substantially enclosed, confined or surrounded by balloons 115 and 120. In block 625 thrombolytic or other treatment may be applied to a selected destination, for example, an occlusion, via a port associated with a drug dispenser 155. Additionally or alternatively, in block 630, thrombolytic treatment may be applied to a selected destination, for example, an occlusion, via a port associated with a vibration mechanism 170, for example, a pump. In block 635, after treatment (e.g., thrombolytic treatment), debris resulting from the occlusion may be extracted using, for example, a suction, at a port, for example port 140 where the treatment was applied. In block 640 an angioplasty procedure and/or stenting procedure etc. may be implemented. Any combination of the above steps may be implemented. Other steps or series of steps may be used.

The foregoing description of the embodiments of the invention has been presented for the purposes of illustration and description. It is not intended to be exhaustive or to limit the invention to the precise form disclosed. It should be appreciated by persons skilled in the art that many modifications, variations, substitutions, changes, and equivalents are possible in light of the above teaching. It is, therefore, to be understood that the appended claims are intended to cover all such modifications and changes as fall within the true spirit of the invention.

1. A catheter device comprising:
a catheter including an inflatable proximal balloon, said proximal balloon being shaped to enable formation of a funnel shape when inflated; and
a guidewire associated with said catheter, said guidewire including at least an inflatable distal balloon.

2. The device of claim 1, comprising a drug dispenser mechanism.

3. The device of claim 1, comprising a vibration mechanism to generate vibrations.

4. The device of claim 1, wherein said proximal balloon and said distal balloon are individually controlled and capable of being located at a selected distance from one another.

5. The device of claim 1, comprising a plurality of ports.

6. (canceled)

7. The device of claim 5, comprising a port to administer the injection of fluids, said port to open a valve and advance the device through the valve in the retrograde direction.

8. The device of claim 5, comprising a port to administer the injection of fluids, said port to extend a vessel and advance the device through the vessel.

9. The device of claim 1, wherein said proximal balloon has a distal end broader than a proximal end when inflated.

10. A treatment method comprising:
inserting a catheter toward a target destination using a guidewire;
inflating a proximal balloon on said catheter, said proximal balloon being disposed closer to a proximal end of said guidewire than a distal balloon on said guidewire is to said proximal end, said proximal balloon being shaped to enable the formation of a funnel shape when inflated; and
inflating the distal balloon on said guidewire, such that said target destination is substantially enclosed by said proximal balloon and by said distal balloon.

11. The method of claim 10, comprising applying treatment to said target destination.

12-14. (canceled)

15. The method of claim 10, wherein said inserting the catheter includes generating vibrations by a vibration source.

16. The method of claim 10, wherein said inserting the catheter includes administering fluids for opening a valve and advancing the catheter through the valve in the retrograde direction.

17. The method of claim 16, wherein opening the valve with a fluid comprises generating a temporarily increased blood flow in an antegrade direction of the valve.

18. The method of claim 10, wherein said inserting the catheter includes administering fluids for expending a vessel and advancing the device through the vessel.

19. The method of claim 10, wherein said inserting the catheter includes administering a substance to said target destination.

20. (canceled)

21. The method of claim 10, wherein said treatment includes generating vibrations toward said target destination.

22. The method of claim 10, comprising forming a treatment area between said proximal balloon and said distal balloon.

23. (canceled)

24. The method of claim 10, comprising performing an angioplasty procedure.

25. The method of claim 10, comprising performing a stenting procedure.

26. The method of claim 10, wherein said proximal balloon has a distal end broader than a proximal end when inflated.

27-44. (canceled)

45. A catheter device comprising:
a catheter including an inflatable proximal balloon, said proximal balloon having a distal end broader than a proximal end when inflated, said distal end of said proximal balloon being farther from a control portion of said catheter than said proximal end of said proximal balloon;
a guidewire associated with said catheter, said guidewire including at least an inflatable distal balloon; and
a vibration mechanism to generate vibrations.

46. (canceled)

47. The device of claim 45, wherein said proximal balloon and said distal balloon are individually controlled.

48. The device of claim 45, comprising a plurality of ports.

49. (canceled)
